(19) United States
(12) Patent Application Publication
(10) Pub. No.: US 2009/0043380 A1
(43) Pub. Date: Feb. 12, 2009

(54) Coatings for promoting endothelization of medical devices

(75) Inventors: Charles Matthew Blaha, San Francisco, CA (US); John To, Newark, CA (US); Loc X. Phan, San Jose, CA (US)

Correspondence Address: Katherine N. Addison Esq., 501 Marie Avenue, Los Angeles, CA, 90042-1305, US

(73) Assignee: SPECIALIZED VASCULAR TECHNOLOGIES, INC., Newark, CA (US)

(21) Appl. No.: 12/128,533

(22) Filed: May 28, 2008

Related U.S. Application Data
(60) Provisional application No. 60/964,142, filed on Aug. 9, 2007.
(60) Provisional application No. 60/993,328, filed on Sep. 11, 2007.
(60) Provisional application No. 61/002,343, filed on Nov. 8, 2007.

(51) Int. Cl. A61F 2/06

(52) U.S. Cl. 623/1.46; 623/1.15; 623/1.42

(57) ABSTRACT

A unique method and coatings are provided for promoting tissue encapsulation of medical devices, especially before antiproliferative drug therapy within a body of a patient in order to prevent excessive restenosis and while avoiding thrombosis (including late stage/stent thrombosis). The method involves delaying the activation of restenosis suppressing (i.e. antiproliferative) drugs in the vicinity of the medical device until a thin layer of geometrically streamlined tissue has deposited itself upon the device. Coatings of one or more layer that provide an aligned scaffolding (i.e. via aligned fibers or aligned grooves) may be used in the method to encourage tissue deposition and/or to delay elution of drug(s) stored beneath or within. The delay phase prior to degradation, erosion, and/or absorption of the coating to release an active drug should last until an optimal amount of controlled restenosis has provided a thin endothelial layer to encapsulate the device.

### BACKGROUND OF THE INVENTION

1. Field of the Invention

The present invention relates to devices and methods for preventing reclosure of a vascular vessel after a surgical procedure therein. More specifically, when the surgical procedure is the implantation of a stent in a coronary vessel, the invention relates to devices and methods for promoting the body's acceptance of the stent, with or without drug elution, by controlling immune responses.

2. Description of the Related Art

Coronary heart disease is a major cause of death in the western world. Most cases of coronary disease involve atherosclerosis in which the heart's vessels become clogged with plaque and fatty deposits to constrict the flow of blood. Modern approaches to restore blood flow and counteract the development of the disease include percutaneous transluminal coronary angioplasty (PTCA) and coronary artery bypass graft (CABG). PTCA is preferably because it is less invasive. However, PTCA alone is frequently unsuccessful in the long-term due to post-angioplasty reclosure of the vessel. Accordingly, common approaches implant long-lasting prosthetics, such as stents, to hold the vessel open after the balloon-tipped catheter used in a PTCA procedure is removed. Modern stents include drugs to address the reclosure problem from both a chemical and a mechanical perspective. Despite the resources that have been devoted to address this problem of post-angioplasty vessel reclosure the current stents are less than perfect and the need for a better solution still exists. (see U.S. Pat. No. (hereinafter USP) 7,223,286 at 2:7-9 and 3:57-58.)

Presently, the two drug eluting stents (DES) on the market that have successfully demonstrated tremendous success in minimizing in-stent restenosis are the Cypher™ (rapamycin) and Taxus™ (paclitaxel) stents. In this way they have proven themselves to be effective. However, both of these stents suffer from the risk of late stage thrombosis (LST) which is a safety problem. So they may be effective but not safe.

It is well documented that the problem with the existing drug eluting stents (DES) is that they prevent the struts from being completely healed over by endothelium and thus can cause thrombosis in the long term. Since stents are foreign body materials, they cause thrombus formation as the body reacts to their exposure in the blood stream. This can lead to rapid occlusion of a blood vessel causing severe complications to the patient as a result. Antiplatelet drug therapy (i.e. using chlopidogrel) is a common way to prevent thrombosis from occurring. When bare metal stents (BMS) are used, oral administration of a systematic antiplatelet drug is typically prescribed for a month after implantation. However, in DES an antiplatelet drug is prescribed indefinitely and can pose a danger to a patient who unexpectedly has to go into surgery. The uncontrollable bleeding encouraged by antiplatelet drugs is a serious risk factor that may even cause a patient to die. Additionally, with DES and orally administered drugs, if a patient forgets to take the drug or cannot afford it, the patient may suffer an ischemic attack or death from stent thrombosis.

Other attempts to reduce the risk of LST utilize different methods and mechanisms for releasing the restenosis-preventing drugs. These include: (i) using different materials [fluoropolymer, phosphorylcholine (PC), polylactic acid (PLA), polyglycolic acid (PGA) combined with PLA, hydroxyapatite (HA), etc . . . ] as matrices to contain the drug, (ii) varying the geometric features of the surface (porous surfaces, micro-wells, micro-holes), (iii) using different types of drugs (Everolimus, Biolimus, Zotarolimus, Tacrolimus), (iv) changing drug release rate profiles, and/or (v) using different type of coatings (PC, collagen) on the stent surfaces to encourage endothelization. None of these approaches have proven effective in eliminating LST while maintaining the high effectiveness in preventing restenosis as Cypher™ and Taxus™. The present invention emphasizes stent coating geometry (i.e. aligned) and drug release rate profile (extended delayed onset followed by rapid pulsatile release).

References in the art refer to a delay coating in the context of a coating that protects and suppresses elution of the drug during the stent implantation phase (see FIG. 2). It is well known in the art to prevent elution of the drug from the stent while the stent is being delivered and positioned within the body. The objective is to avoid systematic loss of the drug before the stent reaches its target location. However, once the stent is in place, the reference art considers the timing appropriate to begin drug elution for a localized effect. The drug eluting stent of the present invention differs from the approaches of the reference art because the coating survives after the placement of the stent in its target position. In the present invention, substantial drug elution does not begin immediately upon stent placement. Rather, the delay coating is used to restrain drug elution both during and after stent placement. According to the present invention, even after the stent is properly situated, the delay coating should continue to prohibit the distribution of the antiproliferative drug for 20-60 days in order to allow sufficient time for beneficial healing and tissue encapsulation of the foreign material and struts that comprise the stent. Nonetheless, in the delayed onset coating of the present invention the initial elution rate of the drug immediately after the stent is implanted need not be zero. Rather, a coating may be considered to be a suitable “delayed onset” coating so long as the initial amount and/or rate of elution is very low compared to a later amount and/or rate.

Physicians typically prescribe antiplatelet drug therapy for the patient with a bare metal stent (BMS) only for 30 days because neointima tend to cover the stent strut in that period and so mask the foreign body from blood (see FIG. 1). Since BMS do not elute any drugs, including restenosis-inhibiting drugs that prevent neointima from developing, the stent struts get covered unlike the situation in most conventional DES. It is well documented that thrombosis (LST) rarely occurs in BMS in the late stage (past 6 months). Neointima helps to smooth out interruptions in the vascular lumen caused by the stent struts which improves hemodynamics. Surface smoothness minimizes stagnate pockets of flow or low velocity/low shear blood flow and this reduces the risk of thrombus formation. The risk with BMS is more likely to be uncontrolled restenosis rather than thrombosis because no long-term antiproliferative drugs are administered locally to bring restenosis to a halt.

Recent research efforts have emphasized the role of the polymer matrix, in which a therapeutic drug is embedded or coated, in causing restenosis and thrombosis. Consequently, product development has focused on eliminating or modifying the composition of the polymer or substituting new drugs.

Based on the assumption that the foreign materials in traditional polymer stent coatings are responsible for producing an immune reaction and late stent thrombosis (LST), the company MIV Therapeutics, Inc. has focused on the design of a polymer-free, bioabsorbable hydroxyapatite coating (see SISM Research & Investment Services article of Apr. 26, 2007 re: MIV Therapeutics, Inc.). Focusing on the chemical composition of the polymer material teaches away from the present invention's solution to the problem. The present invention focuses on abolishing the unstructured, poorly designed, and/or biologically incongruous geometry of conventional scaffolds. The intravascular scaffold can be a highway to natural endothelization or a roadblock, depending upon the uniformity, alignment, and orientation of the constituent materials (i.e. fibers) of which it is composed.

Another company, Conor Medsystems, Inc. (acquired by Johnson & Johnson) directed its efforts to the controlled drug delivery process. However, the drug wells of the Conor CoStar™ stent took the form of dots rather than channels. These wells were neither longitudinally aligned nor continuous. Thus, the failure of the CoStar™ stent is suspected to be due, in part, to the inability of the spotted reservoir system to encourage structured endothelization.

These activities overlook the fact that even non-polymer coated drugless stents, known as bare metal stents (BMS), cause thrombosis without antiplatelet treatment immediately post implantation and cause restenosis long-term even with antiplatelet treatment. Antiplatelet drugs are not necessarily also restenosis-suppressing antiproliferative drugs and, regardless, they are not administered long-term following BMS implantation.

During stent placement, damage to mural tissue bordering the vessel lumen instigates an immune response. Popular traditional and current approaches to preventing restenosis characterize this immune response as something to be avoided. Current methods for avoiding the immune response that causes restenosis are directed at formulating more biocompatible stent coatings and drugs. These approaches and methods do not adequately address late stent thrombosis. In contrast, the present invention recognizes the beneficial value of a controlled immune response and provides a stent to work with the natural response rather than trying to avoid it by burying the stent with coatings and drugs to suppress it. The objective of the present invention is to provide a stent capable of eliminating both detrimental (uncontrolled) restenosis and thrombosis (both initially and at later stages, i.e. after six months of stent implantation). This avoids the current tradeoff that must be made between the two equally important goals ((i) no restenosis, (ii) no thrombosis) required by the choice between BMS and conventional DES.

When conventional BMS (i.e. without drugs or an aligned coating) are implanted, the new endothelium that develops is typically dysfunctional and does not effectively inhibit restenosis. This dysfunctional endothelium causes problems in the long term post-implantation in the form of uncontrolled restenosis. Thus, the practice of eluting antiproliferative drugs from stents to inhibit restenosis during the initial post-implantation period developed. The endothelium that develops on unaligned stents is dysfunctional because the non-aligned struts do not merge well with the naturally aligned elongated endothelial cells (ECs) and proteins traversing a healthy blood vessel. It is easier for non-endothelial cells to form upon an unaligned, unstructured stent than it is for endothelial cells to integrate themselves. Therefore, the cells that grow to become the new endothelium are not true endothelial cells and that is at least part of the reason why the post-implantation in vivo “endothelial” layer formed on conventional (unaligned) stents is dysfunctional.

Some references disclose the “in vivo” adherence of endothelial cells to the surface of the stent (i.e. see U.S. Pat. No. (hereinafter USP) 7,037,332 of Kutryk, et al. and assigned to Orbus Medical Technologies, Inc.). The Kutryk (USP '332) patent discloses an antibody in a coating on a medical device that reacts with a surface antigen of natural endothelial cells to induce their adherence to the device. Kutryk relies upon a surface antigen rather than aligned fiber geometry to induce endothelization.

Some references, such as U.S. Pat. No. 6,855,366 by Smith, et al. (and assigned to the University of Akron) acknowledge some of the advantages of nitric oxide delivery using nanofibers. However, the Smith patent is limited to fibers of poly(ethylenimine). Further, Smith does not recognize: (i) the importance of aligning the fibers to facilitate functional endothelization, nor (ii) the possibility of using fibers as a coating to delay the onset of drug release for drugs other than nitric oxide.

### BRIEF SUMMARY OF THE INVENTION

The present invention presents medical devices and methods for their operation such that the devices will be accepted by the body in the short-term and the long-term. By controlling the body's immune response (i.e. as manifested in thrombosis and restenosis), the invention disguises the stent in vivo by the body's own tissue. The tradeoff between short-term and long-term benefits and between the advantages of conventional bare metal stents (BMS) and contemporary drug-eluting stents (DES) are avoided as the present invention combines the advantages of both stent types and to provide immediate and enduring benefits.

The present invention realizes the value in letting some natural immune response reactions occur. According to the principles of the present invention, some restenosis is desirable because restenosis can cover the stent struts. Once the stent struts are smoothly covered, a more harmful immune response (late stent/stage thrombosis or LST) can be suppressed because there is no issue with hemodynamics. No stent coating is more biocompatible than one made in vivo, from naturally synthesized biomaterials such as the tissue generated by restenosis. The creation of natural coatings in vivo avoids an aggravated immune response by the body, thereby preventing inflammation, excessive restenosis, clotting, smooth muscle cell migration and proliferation, hyperplasia, and thrombosis.

### BRIEF DESCRIPTION OF THE SEVERAL VIEWS OF THE DRAWINGS

FIG. 1 shows a restenosis cascade indicating at what point in time following the implantation of a stent various biological activities have occurred. The invention provides for elution of a restenosis suppressing drug (which will bring such biological activities to a halt) anywhere from 5-60 days following stent implantation.

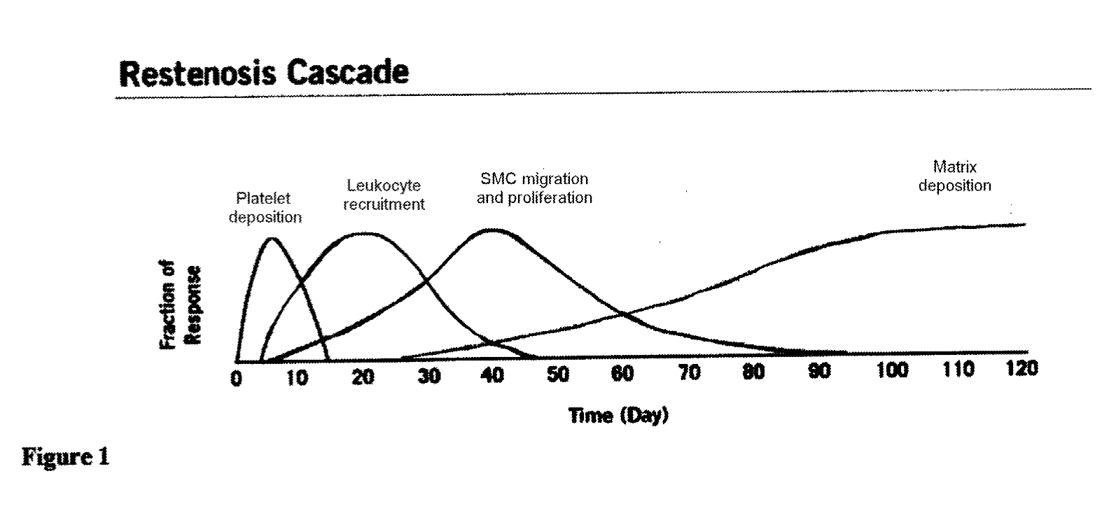

FIG. 2 shows the cumulative amount of drug released from popular drug eluting stents in the days following stent implantation as compared with the stent of the present invention. FIG. 2 demonstrates how the delay coating on the stent of the present invention virtually completely suppresses drug release until approximately 25 days after the stent is properly positioned, in contrast to conventional DES that only slow the rate of release. The Taxus™ DES provides a steady slow release rather than a delayed pulse release.

FIG. 3 is a side cross-sectional view of the stent struts (zig-zag or sinusoidal in shape) and aligned fiber coating with the aligned fibers in a staggered pattern connecting adjacent struts. The struts provide radial columnar strength and support while the aligned fibers provide longitudinal flexibility.

FIG. 4 shows the initiation of drug elution from a drug matrix upon a stent strut after degradation of a delay coating.

FIG. 5 shows a protective hydrophilic layer sandwiched between the amphipathic (weak polar, partly hydrophobic) drug matrix layer and the amphipathic (weak polar, partly hydrophobic) outer layer to create a delayed onset, sudden pulsatile release of the drug.

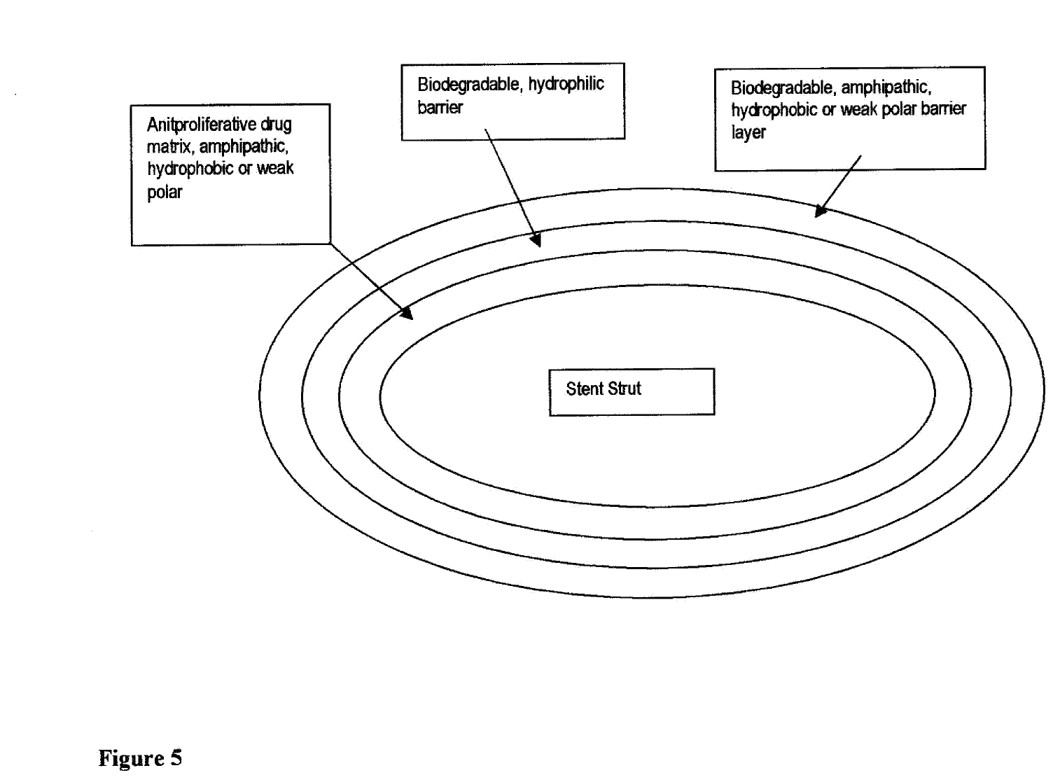

FIG. 6 shows another embodiment in which protective hydrophilic materials are distributed in pockets within the amphipathic (weak polar, partly hydrophobic) outer layer adjacent to the drug matrix layer to create a delayed onset, sudden pulsatile release of the drug.

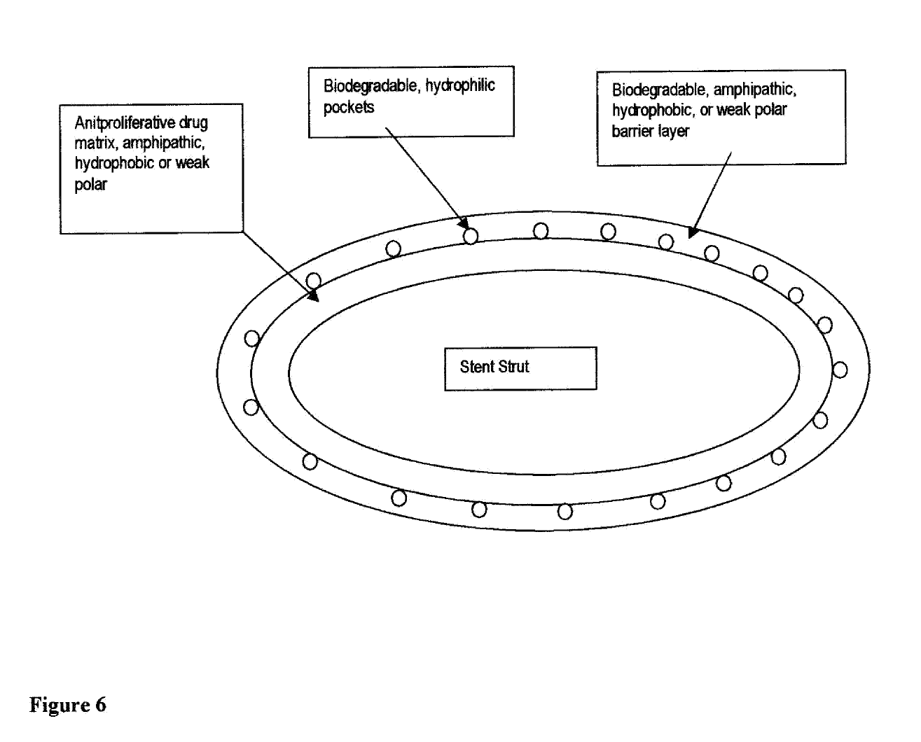

### DETAILED DESCRIPTION OF THE INVENTION

In the simplest form of the present invention, a biodegradable layer is designed to act as a switch to turn on the release of antiproliferative drug (i.e. rapamycin, paclitaxel) once enough proliferation has occurred to encapsulate the stent strut. This can be achieved by timing the switch to match the typical time (Encapsulation Development Time) for development of tissue encapsulation (timing approach) or to have the encapsulation event itself trigger the switch (event triggered approach).

Under the timing approach, a biodegradable layer can be coated on the drug matrix that would degrade enough to allow drug elution around 20 to 40 days, the typical time of tissue encapsulation of a stent strut. For the switch to be effective, it must effectively block antiproliferative drugs from eluting for the duration of Encapsulation Development Time and then quickly turn on to fully elute the drug.

Since the typical antiproliferative drug (i.e. rapamycin, pacitaxel, etc.) is hydrophobic, a good solid first barrier layer should be made of a hydrophilic, biodegradable substance such as polyvinyl alcohol, polyethylene glycol, gelatin, dextran, pullulan, and/or salts (NaCl, DMSO). A second barrier layer of a more hydrophobic substance can be coated over this first hydrophilic barrier to control the degradation time to better match the Encapsulation Development Time. This outer barrier layer of a more hydrophobic substance can be selected from polylactic acid (PLA), polyglycolic acid (PGA), a copolymer of PLA and PGA (PLGA) or polycaprolactone (PCL), other biodegradable polyesters, collagen, polyamino acids, or other hydrophobic, biodegradable polymers.

Under the event triggered approach, there are several ways to trigger the switch to allow drug elution to occur upon tissue encapsulation of the stent strut:
1. First, the coating covering the drug matrix is designed to immediately break down to allow drug elution upon tissue encapsulation. This can be achieved by coating the drug matrix with a slightly to hydrophobic, biodegradable layer that breaks down quickly upon presence of a slightly to hydrophobic environment such as restenotic material. A thin layer of wax or a fatty substance exemplify the type of coating to be used. Examples of these include lipoprotein, collagen, polyamino acids, PLA, PLGA, and polycaprolactone,
2. Second, the drug matrix (material in which the drug is embedded rather than coated) itself can be hydrophobic, biodegradable such that it degrades quickly when exposed to the hydrophobic environment created by restenotic tissue.
3. Third, the antiproliferative drug can be bound to a molecule that inactivates the drug until restenosis factors (i.e. collagen, proteoglycans) are present.
4. The switch can be turned on by other factors accompanying tissue encapsulation including: hormones, enzymes, and/or peptides, etc.
5. Pressure can be used to induce release of the drug, i.e. by housing the drug within a semi-permeable membrane that bursts.
6. pH changes can be used to induce release of the drug if the material coating the drug is sensitive to acids or bases and degrades upon being subjected to acidic or basic environments. In one embodiment, the drug is coated with a slightly hydrophobic, acid-sensitive layer of PLGA. Tissue encapsulation of the stent strut can trap the PLGA and the acids produced from PLGA degradation. Subsequently, the concentration is dramatically increased and leads to rapid degradation of the PLGA itself.

This event triggered approach offers a high degree of control of drug elution and/or activation. The onset of drug elution and/or the catalyst for drug activation is particularized to occur independently and exclusively on the stent localities encapsulated by tissue while the elution is restrained and/or the drug remains dormant and inactive on the stent localities that are still bare and unencapsulated. Encapsulation rates vary between procedures, individuals, and stent localities. Therefore, event-triggered drug control provides an individualized approach for enhanced accuracy, safety and effectiveness.

In one embodiment, the present invention uses aligned nanofibers and/or aligned nanogrooves to form the stent coating to create an artificial functional endothelial layer that will attract the deposition of a natural endothelial layer. The natural endothelial layer is composed of aligned, elongated endothelial cells that will align themselves amongst the aligned fibers and deposit directly on the stent itself even when the aligned nanofiber coating is not loaded with any specifically reactive linking agents.

In contrast, the Kutryk patent (USP '332) only discloses amorphous carbon, fullerenes and hollow nanotubes (rather than aligned rod-like nanofibers) for the matrix material of a stent. Kutryk relies upon specific components, antibodies, to react with specific, known antigens in natural endothelial cells to create the first endothelial cell layer without any specific cell orientation. That is, the device, coating and methods of Kutryk “may stimulate the development of an endothelial cell layer with random cell orientation on the surface of the medical device” (see USP '332 at 4:26-31) but they do not themselves serve as an aligned functional endothelial cell layer.

The xenographic/xenogenic artificial functional endothelial layer of aligned fibers and/or aligned grooves may be composed of or seeded with synthetic materials, allogeneic materials (cells or clones from a second subject of the same species as the patient), and/or heterologous materials (cells or clones from a second subject not of the same species as the patient). In any case, the aligned geometry of the artificial functional layer paves the way for the growth of a natural functional layer of autologous endothelial cells produced in vivo that will encapsulate the stent struts and injured to tissue to a depth of 0.1 mm thereby masking its xenographic (foreign) nature to preclude an immune response that may cause thrombosis.

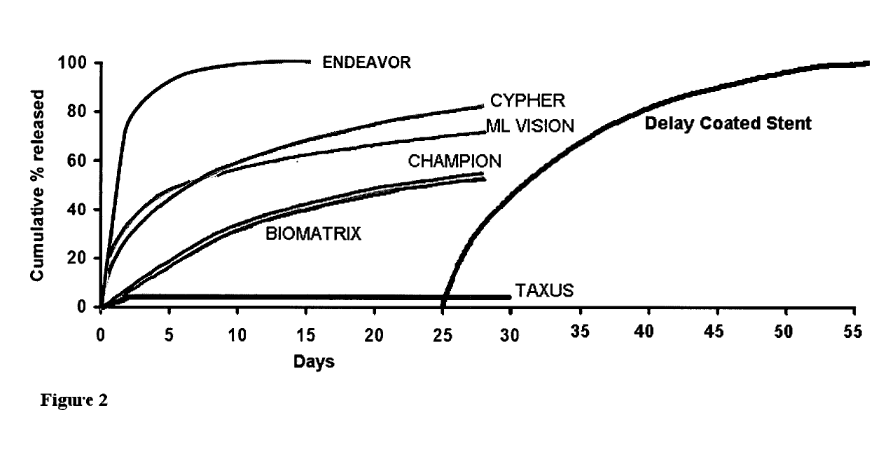

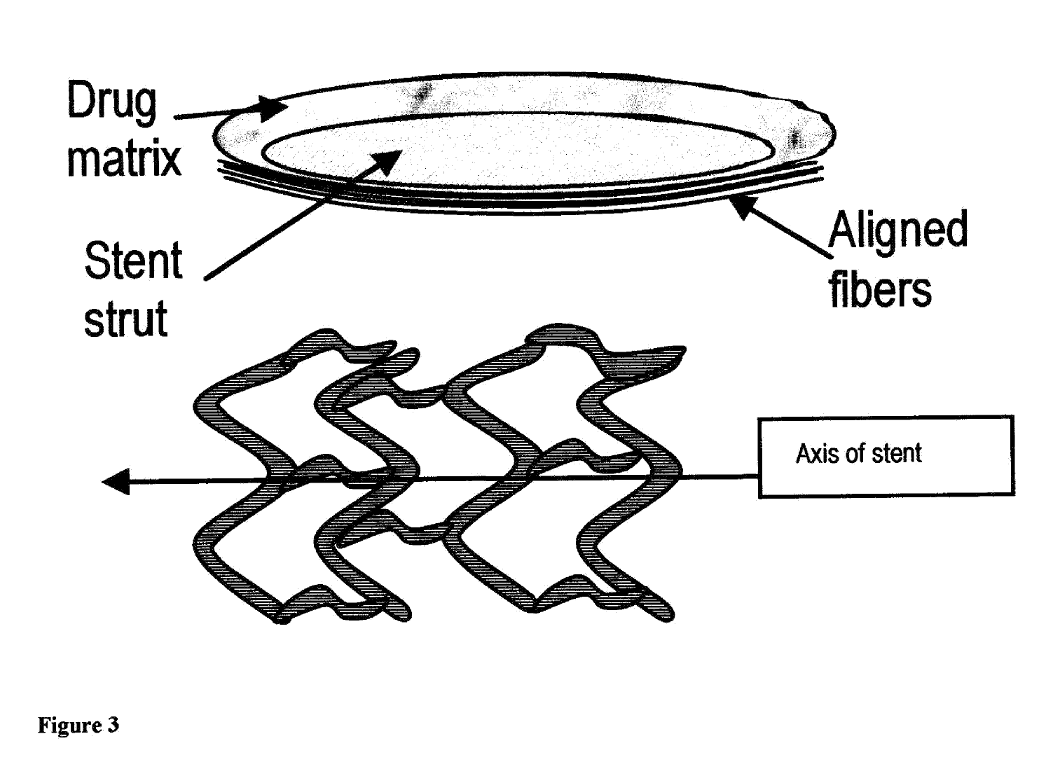

The present invention is a novel approach to solving the problem of LST without sacrificing the effectiveness of the antiproliferative drug in preventing restenosis. This is done by depositing a biodegradable layer of aligned microfibers (AMF), aligned nanofibers (ANF), and/or aligned grooves (AG) on top of a DES as an effective means to delay the onset of antiproliferative drug release as well as to facilitate endothelization (see FIG. 2 and FIG. 3). This way the patient benefits from two desired characteristics:
1. the safety of the BMS by having a smooth endothelium or neointima encapsulating the stent struts; and
2. the long term effectiveness of proven DES such as Cypher and Taxus by maintaining delivery of a local antiproliferative drug from the stent but with a delayed onset.

The AMF/ANF/AG material may take the form of a coating, a matrix, or a stent body so long as its structure and orientation are such that it can both facilitate endothelization and also delay the onset of drug release, if drugs are used. Preferably, the AMF/ANF/AG material lasts for 15-30 days before it is fully degraded to expose the drug underneath. However, it may work by fully degrading anywhere between 5-60 days. The AMF/ANF/AG material is preferably made of PGA or a copolymer of PGA-PLA. These are proven compounds used on DES as well as biodegradable sutures and are well documented for their compatibility with blood. PGA and PGA-PLA are especially well suited to degrade within 15-30 days. The delay time before onset of release of the antiproliferative or immunosuppressant drug (i.e. rapamycin, paclitaxel, everolimus, etc.) is equal to the time it takes the AMF/ANF/AG material to fully degrade. This delay time is controlled by the exact chemical compounds used to create the coating and also the thickness. For example, since 50% PLA:50% PGA degrades more quickly than a 75%PLA:25% PGA mix, to obtain the same drug release onset delay a thicker layer of 50% PLA:50% PGA would be used than if a 75%PLA:25% PGA mix were used. The AMF/ANF/AG material is preferably between 0.1 micron and 20 microns thick.

Alternatively, instead of PGA and/or PLA, the AMF/ANF/AG material can also preferably be made of poly(ethylene glycol) (PEG), also known as poly(ethylene oxide) (PEO) or polyoxyethylene (POE). Caprolactone (CPL) can also be used. CPL and PEG are elastomeric materials and if the AMF/ANF/AG medical device has elastomeric properties it will better conform to the natural shape of the lumen in which it is inserted or implanted. Elastomeric materials are better able to close gaps between a stent wall and a lumen wall. Avoiding incomplete apposition of the stent struts against the lumen wall reduces the formation of stagnant pockets in which a thrombus is more likely to develop. Metallic stent struts are typically stiff and cannot conform well to the lumen when the lumen is not smooth and uniform, as is often the case. However, an elastomeric coating upon non-elastomeric stent struts ameliorates this problem by flexing, bending, expanding, and contracting to occupy the differential spaces created by the nonconformity between the lumen wall and the stent struts. Alternatively, if the stent struts themselves are made of AMF/ANF/AG elastomeric materials they can directly model the irregular surface patterns of anatomic lumens.

The AMF/ANF/AG material can also be made out of biological molecules (biomolecules) such as collagen, fibrin, or fibrinogen. Various other substances that can be used to form the AMF/ANF/AG material are: phosphorylcholine, nitric oxide, high density lipoprotein, polyzene-F, PTFE polyetherester, hydroxyapatite, polyhydroxy-butyrate, polycaprolactone, polyanhydride, poly-ortho ester, polyiminocarbonates, polyamino acids, and polyvinyl alcohol.

Irrespective of the chemical components used to form the AMF/ANF/AG material, when used as a delay coating the AMF/ANF/AG material is preferably negatively charged and also preferably has a nitric oxide functional group. Thus, as the fibers degrade, nitric oxide is released. Within the bloodstream of the lumen occupied by the stent, the nitric oxide serves to further inhibit restenosis by preventing platelet aggregation and macrophage/leukocyte infiltration, reducing smooth muscle cell proliferation, and decreasing inflammation generally while aiding the healing process. An aligned coating with a nitric oxide group (ANO) on a stent (or other intravascular medical device) forms an artificial endothelium layer due to the smooth, streamlined surface the aligned fibers/grooves provide coupled with the ability of nitric oxide to prevent aberrations on this smooth surface as the fibers degrade.

The present invention recognizes the use of any biocompatible materials that can be formed into aligned nanofibers, aligned microfibers, or aligned grooves for the AMF/ANF/AG material used to form a stent, a coating, or a matrix for drug(s). The present invention also recognizes the ability to use the AMF/ ANF/AG material in conjunction with other coatings, layers, matrices, pores, channels, reservoirs, etc. to delay onset of the release of any therapeutic drug and/or to encourage structured (i.e. aligned) endothelization.

The present invention also teaches the criticality of matching the time period of delay prior to drug release with the time it takes for the AMF/ANF/AG stent surface to become covered (i.e. encapsulated) by endothelization to a depth of approximately 0.1 mm. The artificial functional endothelium layer itself is a very thin (i.e. only one or a few cells thick). A thin layer does not burden the stent with unnecessary volume (i.e. on the periphery of a cross-section) that could make insertion and adjustment within the lumen more difficult. A thin layer also does not significantly reduce the inner diameter of the stent's lumen and therefore does not interfere with hemodynamics or obstruct blood supply to a treated area.

When the stent is not formed of a material (i.e. such as an elastomeric aligned material) that enables it to conform to the shape of a lumen surface, a thrombus is more likely to develop causing a localized inflammatory reaction. Also, when the stent doesn't conform well to the shape of a lumen, the process of restenosis cannot be effectively controlled. Although systematic drugs administered with BMS and drugs supplied by DES can slow or modulate the rate of ineffective restenosis they are not typically used to encourage a moderate amount of beneficial restenosis. Any restenosis that does occur in a vessel having an uneven surface with stent struts that inadequately conform to the natural cell and protein structure (and/or shape) of the vessel is likely to be uncontrollable and problematic. Smooth muscle cell migration and proliferation is likely to form the first tissue layer over the stent struts. In contrast, the present invention provides a pre-formed artificial functional endothelial layer to provoke a first in vivo layer of natural endothelial cell growth.

According to the present invention, an aligned (i.e. AMF/ANF/AG/ANO) coating on the luminal surface aligns both the blood flow and the growth of natural endothelial cell layers in a uniform, optimal direction (i.e. longitudinally along the central axis of the lumen). An aligned inner coating accelerates and optimizes blood flow for better drainage and support. Normal blood flow around the stent flushes out immune response agents and toxins, as they are produced, to accelerate drainage and healing. Normal blood flow also feeds the developing, natural endothelial cell layer above the artificial functional endothelial stent coating with nutrients.

Once the natural endothelial cell layer has developed to a sufficient extent (i.e. a depth of approximately 0.1 mm) and moderate amounts of beneficial (i.e. aligned) restenosis have been permitted to occur, the result is a camouflaged stent buried within normal, healthy tissue. No foreign materials are detectable by the blood and so the blood related immune response and inflammation are inhibited, thereby greatly reducing the risk of thrombosis. As drugs begin to be eluted from DES upon degradation of the aligned coating, the beneficial, controlled restenosis process (“encapsulation”) comes to a halt. The stent remains stably buried but the thickness of the luminal walls stops increasing to avoid reclosure. The drugs are powerful enough to prevent additional encapsulation but cannot undo the beneficial, stent-sealing, encapsulation that has already occurred.

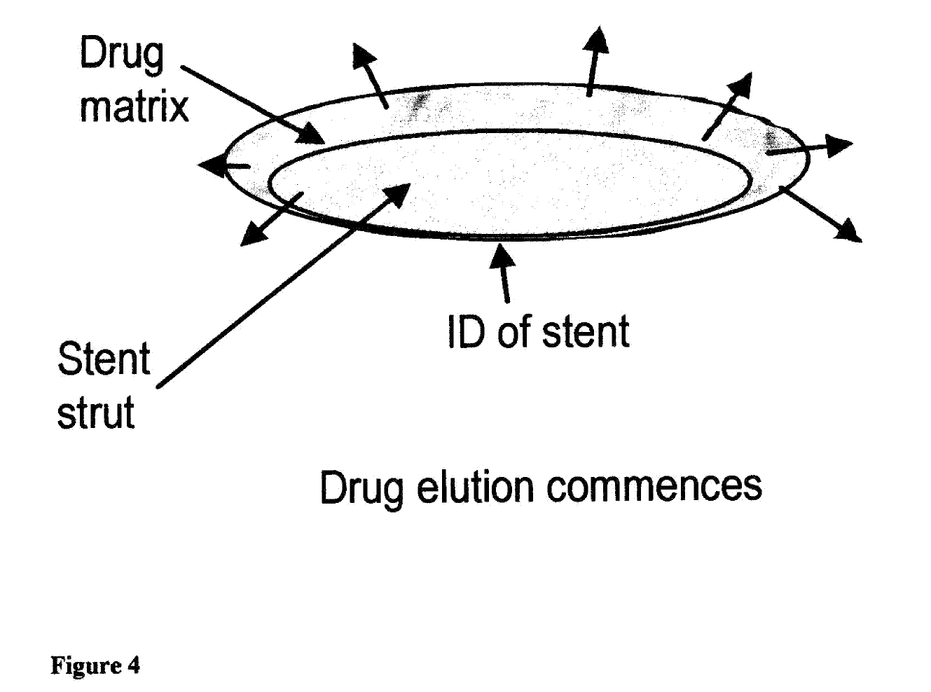

Elution of the therapeutic antiproliferative or immunosuppressant drugs will arrest the proliferation of neointima (smooth muscle proliferation and protein deposition) (see FIG. 4). Due to the delay in the onset of drug release, by the time the drugs are released all the stent struts are encapsulated with endothelium and/or smooth muscle. Therefore, higher dosages of drugs, faster elution rates, and/or more aggressive drugs can be used to ensure maximum effectiveness in preventing restenosis in the long term without fear of LST from an immune reaction. Once the stent struts are smoothly buried beneath a thin natural tissue layer thrombosis is unlikely.

Optionally, the stent may have semi-permeable cross-sectional side walls extending through the surface area of the cross section on each end adjacent to a target site to be treated with an eluted drug. The side walls would serve as barriers to the drug to concentrate it at the target site and avoid the negative effects of systematic drug distribution. Such sidewalls would also conserve the drug to be maintained where it is needed most to allow less total drug within the stent to be equally effective by reducing the washout effect. Reducing the total drug stored in the state (while maintaining effectiveness) is beneficial because then the stent walls can be thinner and it is also less expensive. The semi-permeable nature of the side walls allows them to permit the influx of important nutrients needed at the constricted vessel site and to permit the outflux of waste thus preserving hemodynamics. The cross-sectional side walls would dissolve naturally in time to correspond with the termination of the desired drug treatment period.

Optionally, the stent may include radioopaque substances in one or more of the materials of which it is formed or in one or more coatings. An array of different, distinguishable radioopaque substances may also be used in each layer or coating. These substances would enable a physician to externally observe the placement, progress, and improvement of the stenting procedure without causing the patient discomfort from an internal inspection and without risking displacing the stent during an internal (i.e. endoscopic) inspection.

Another approach to avoiding LST while still controlling restenosis is by accelerating the endothelization of the stent through aligned scaffolding without the antiproliferative drug. The bare stent can be made of (at least in part) or coated with elongated AMF/ANF/AG/ANO aligned with the direction of blood flow (i.e. long axis of fibers parallel to the direction of blood flow). Endothelial cells (ECs) are themselves elongated and tend to also be aligned with the direction of blood flow. By aligning the fibers with the preferred alignment of ECs, the deposition of ECs over the stent (including but not limited to the stent struts) is accelerated (aligned scaffolding). The presence of ECs tends to arrest the restenosis process (smooth muscle proliferation). The AMF/ANF/AG/ANO are preferably laid down on the inner diameter (ID) of the stent (see FIG. 3). The outer diameter (OD) or abluminal surface of the stent is typically embedded in or aligned against the luminal surface of the vessel so that the longitudinal alignment of the fibers here is not as important as for the inner diameter or luminal surface of the stent.

The stent struts are typically 50 to 100 microns wide. The fibers are preferably 0.5 to 10 microns wide. Therefore, regardless of the stent strut orientation, the fibers can have an aspect ratio of 5 or greater. By having an aspect ratio greater than 2, the fibers can provide effective longitudinally aligned scaffolding for ECs to grow on.

The AMF/ANF/AG/ANO coating or surface can be impregnated or coated with antiplatelet or anticoagulant drugs such as heparin, ticlopidine, chlopidrel, enoxaparin, dalteparin, hirudin, dextran, bivalirudin, argatroban, danparoid, Tissue Factor Pathway Inhibitor (TFPI), GPVI antagonists, antagonists to the platelet adhesion receptor (GPlb-V-IX), antagonists to the platelet aggregation receptor (GPIIb-IIIa) or any combination of the aforementioned agents.

The AMF/ANF/AG/ANO material can also be impregnated with endothelization promoting substances such as vascular endothelial growth factor (VEGF), angiopoietin-1, antibodies to CD34 receptors, and/or hirudin, dextran.

The coating can be applied to the inner diameter (ID) of the stent in the form of longitudinally aligned microfibers, nanofibers, grooves, or nitric oxide carrying elements by several modified processes of electrospinning:

1A. Aligned Nanofibers on stent struts only: A dispensing syringe is loaded with a solution of the fiber material and is charged (i.e. positive or negative, preferably negative) with a high voltage (>1 kV) to charge the solution. The stent is either grounded or charged by applying the opposite voltage (i.e. preferably positive). The outer diameter (OD) of the stent is covered with a polar or conductive tube that sticks to the fiber material well. For example, if PGA or PLA are used as the polymer solution from which the fiber material is formed, polyethylene terephthalate (PET) is heat shrunk on the OD of the stent. The stent is held by a grounded or charged (i.e. preferably positive) collet on the OD of one end. The dispensing syringe needle with a 90 degrees bend (or side hole) at the tip is inserted inside the ID of the stent from the open end of the stent. The charged solution is dispensed from the needle tip onto the stent ID as longitudinally aligned micro/nanofibers/grooves/nitric-oxide carrying elements as the syringe tip is moved back and forth longitudinally. As the syringe tip completes one pass from one end to the other, the collet is indexed (turned incrementally) to lay down the adjacent fiber. This process continues until the whole stent ID is covered with aligned fibers, grooves or elements. Once the coating is finished, the cover (i.e. polar or conductive tube such as PET) on the OD can be peeled off to clear the stent openings of fibers.

1B. Aligned Nanofibers covering all stent: A dispensing syringe is loaded with a solution of the fiber material and is charged (i.e. positive or negative, preferably negative) with a high voltage (>1 kV) to charge the solution. The stent is either grounded or charged by applying the opposite voltage (i.e. preferably positive). The stent is held by a grounded or charged (i.e. preferably positive) collet on the OD of one end. The dispensing syringe needle with a 90 degrees bend (or side hole) at the tip is inserted inside the ID of the stent from the open end of the stent. The charged solution is dispensed from the needle tip onto the stent ID as longitudinally aligned micro/nanofibers/grooves/nitric-oxide carrying elements as the syringe tip is moved back and forth longitudinally. As the syringe tip completes one pass from one end to the other, the collet is indexed (turned incrementally) to lay down the adjacent fiber. This process continues until the whole stent ID is covered with aligned fibers, grooves or elements.

2. The highly charged (i.e. −10 kV) syringe as described above is fixed longitudinally. The stent is grounded. A ring of opposite charge (i.e. +10 kV) is placed near the stent. The dispensing syringe is pulsed by pulsing syringe pressure, a needle valve, or charging to completely dispense one aligned fiber. The stent is then rotationally indexed for the next pulsed dispensing.

3. A hollow ring containing the solution of fiber material has series of micro/nano-holes on the end for dispensing parallel fibers arranged in a diameter close to the diameter of the stent. The ring is highly charged (i.e. −10 kV) to charge the fiber material in solution. The stent is grounded. A ring close to the diameter of the stent is charged with an opposite charge (i.e. +10 kV) on the opposite end of the stent. This charged state will cause the solution which forms the fibers to eject from the holes in parallel, longitudinally towards the oppositely charged ring while simultaneously adhering to the stent along the path from one ring to another.

In another embodiment, the inner surface of the stent strut can have micro/nano-grooves etched on it longitudinally (parallel to axis of stent). ECs will tend to grow into these grooves. The grooves are preferably 1 to 10 microns wide. In the same manner, the grooves can also be ridges or channels. The longitudinally aligned micro/nano-grooves may also be used as reservoirs or longitudinal wells for storing therapeutic drugs within the aligned fiber layers for controlled or multi-phase elution.

These AMF/ANF/AG/ANO stents are particularly advantageous when applied to intravascular bifurcations or vessels with one or more corollary branch adjacent to a main lumen. Bifurcated vessels tend to have much higher rates of restenosis with both conventional BMS and DES than do non-bifurcated vessels.

The present invention controls tissue encapsulation of the stent and of injured tissue in at least three ways: biologically, geometrically, and chronologically.

Biologically, aligned nano/microfibers with or without aligned nano/microgrooves therein (or alternatively, aligned grooves formed within a non-fibrous material) facilitate functional endothelization by encouraging a uniform orientation in any cell growth that occurs (whether of true endothelial cells or artificial endothelial cells). The polymers or other materials chosen for the construction of the nano/microfibers or nano/microgrooves must be biocompatible to permit the natural flow of blood and other bodily fluids through the lumen adjacent the stent's inner surface without elicitation of an immune response or thrombosis. The materials used to form the fibers or the material within which the grooves are etched can be synthetic or naturally derived. Suitable materials include: biodegradable materials such as polyglycolic acid (PGA), polylactic acid (PLA), copolymer of PLA and PGA (PLGA), hydroxyapatite (HA), polyetherester, polyhydroxybutyrate, polyvalerate, polycaprolactone, polyanhydride, poly-ortho ester, polyiminocarbonates, polyamino acids, polyethylene glycol, polyethylene oxide, and polyvinyl alcohol; non biodegradable polymers such as fluoropolymer like polytetrafluoroethylene (PTFE), polyzene-F, polycarbonate, carbon fiber, nylon, polyimide, polyether ether ketone, polymethylmethacrylate, polybutylmethacrylate, polyethylene, polyolefin, silicone, and polyester; biological substances such as high density lipoprotein, collagen, fibrin, phosphorylcholine (PC), gelatin, dextran, or fibrinogen.

Geometrically, the invention is designed to only allow 0.1 mm thickness of encapsulation (of stent struts or the entire stent body and of injured tissue) before the drug elution process begins to inhibit further encapsulation. Another aspect of geometric control is the alignment of fibers/grooves and all growth thereupon whether it be endothelial cells, smooth muscle cells, proteins, matrix fibers, or collagen fibers. Due to the structure supplied by the fibers/grooves, all subsequent in vivo growth, migration, and/or proliferation is necessarily aligned to correspond to the template set by the fibers/grooves. Aligned growth does not interfere with blood flow. Further, even if the initial natural layers of biologically derived materials deposited are not the ideal materials (i.e. smooth muscle cells instead of endothelial cells), as long as they are aligned they are suspected not to impede the deposition of the optimal materials when they come along.

Chronologically, the invention assures that the complete degradation of the polymer (or other material) layer serving as a delay coat for the antiproliferative drug corresponds to the time when an optimal amount (i.e. 0.1 mm thickness) of encapsulation has occurred because that point in time also marks the onset of elution of the antiproliferative drug which will suppress further thickening of tissue encapsulation. Temporal control over the elution of the antiproliferative and/or other therapeutic drugs may also be achieved by an external activation means that signals for the aligned drug reservoirs to begin elution. The external activation means may be electromagnetic radiation, infrared light, microwave radiation, x-ray radiation, etc. This type of external activation means would provide very precise control of the onset of drug elution. Since the rate of encapsulation will vary from individual to individual and from procedure to procedure depending upon a multitude of factors, a pre-elution assessment (i.e. imaging for endothelial cell markers) of the extent of encapsulation can precede initiation of the external activation means to ensure elution does not begin prematurely.

The materials and dimensions described here are not meant to be limiting. The general concept can be extended to other specific embodiments or ranges.

From the above description of the invention, those skilled in the art will perceive improvements, changes and modifications. Such improvements, changes and modifications within the skill of the art are regarded as covered by the appended claims directly or as equivalents.

What is claimed:

1. A coating, on a medical device, configured to promote formation of a protective matrix layer of a body's own tissue in situ and in vivo.

2. The coating of claim 1, on a drug eluting medical device, configured for delaying onset of elution of a restenosis suppressing drug until the device has been encapsulated by a thin layer of the body's own tissue, wherein the coating promotes tissue encapsulation, encourages tissue proliferation, and facilitates controlled restenosis.

3. The coating of claim 2, comprising a combination of a biodegradable, hydrophilic barrier adjacent to one or more site of drug storage and a biodegradable, slightly hydrophobic barrier adjacent to the hydrophilic barrier.

4. The coating of claim 3, wherein the hydrophilic barrier comprises at least one element selected from the group consisting of: dextran, polyvinyl alcohol, polyethylene glycol (PEG, also known as poly(ethylene oxide) (PEO) or polyoxyethylene (POE)), gelatin, pullulan, heparin, hirudin, ticlopidine, chlopidogrel, a salt, and an anticoagulant.

5. The coating of claim 3, wherein the slightly hydrophobic barrier comprises at least one element selected from the group consisting of: polylactide, polylactic acid, polyglycolide, polyglycolic acid, polylactide-polyglycolide, polycaprolactone, polyamino acid and any copolymer of the aforementioned elements.

6. The coating of claim 3, comprising at least two layers, wherein one layer comprises the hydrophilic barrier and a separate layer comprises the slightly hydrophobic barrier.

7. The coating of claim 3, wherein the hydrophilic barrier is distributed in pockets within the hydrophobic barrier which forms a matrix or coating.

8. The coating of claim 7, wherein the hydrophobic barrier matrix or coating has a higher viscosity than the hydrophilic barrier pockets.

9. The coating of claim 7, wherein the hydrophilic barrier pockets repel the drug.

10. The coating of claim 7, wherein the hydrophilic barrier pockets absorb water quickly upon degradation of the hydrophobic barrier matrix or coating to form a low viscosity solution to facilitate drug elution.

11. The coating of claim 10, wherein absorption of water by the hydrophilic barrier pockets upon initial degradation of the hydrophobic barrier matrix or coating causes water to flood the hydrophobic barrier, inducing its hydrolysis and accelerating further degradation of the hydrophobic barrier.

12. The coating of claim 7, wherein the hydrophilic barrier consists of dextran and the hydrophobic barrier consists of 75% polylactic acid and 25% polyglycolic acid.

13. The coating of claim 1, comprising aligned fibers.

14. The coating of claim 1, comprising aligned grooves.

15. The coating of claim 13, wherein the medical device is a stent.

16. The coating of claim 14, wherein the medical device is a stent.

17. The coating of claim 2, further comprising a second coating wherein the second coating is a protective coating.

18. The coating of claim 13, wherein the aligned fibers are nanofibers or microfibers having diameters from 0.5 to 10 microns wide and having lengths at least twice the size of the diameters.

19. The coating of claim 14, wherein the aligned grooves are nanogrooves or microgrooves having diameters from 0.5 to 10 microns wide and having lengths at least twice the size of the diameters.

20. The coating of claim 13, wherein the fibers comprise a nitric oxide functional group and release nitric oxide as they degrade.

21. The coating of claim 18, wherein the aligned fibers are oriented at an angle of 0 to 30 degrees relative to a long axis of the medical device.

22. The coating of claim 19, wherein the aligned grooves are oriented at an angle of 0 to 30 degrees relative to a long axis of the medical device.

23. The coating of claim 13, wherein the aligned fibers are positioned on an inner surface, or a luminal wall, of the medical device and are aligned approximately parallel to a long axis of the medical device.

24. The coating of claim 13, wherein the aligned fibers are positioned on an outer surface, or an abluminal wall, of the medical device and are aligned approximately parallel to a long axis of the medical device.

25. The coating of claim 13, wherein the fibers form an artificial endothelium that aligns both blood flow and growth of endothelial cells in a uniform direction to facilitate rapid development of a functional endothelium.

26. The coating of claim 25, wherein the functional endothelium is a one cell layer that inhibits proliferation of smooth muscle, also termed restenosis, by releasing nitric oxide.

27. The coating of claim 15, wherein the stent has struts, and the aligned fibers are only positioned on inner and outer surfaces of the struts of the stent.

28. The coating of claim 15, wherein the stent has struts, and the aligned fibers cross at least two struts of the stent.

29. The coating of claim 1, wherein the coating is biodegradable, bioabsorbable, or bioerodable.

30. The coating of claim 29, further comprising a layer of aligned fibers, wherein the layer of aligned fibers is nonbiodegradable, nonbioabsorbable, and nonbioerodable.

31. The coating of claim 1, wherein the coating is sufficiently elastomeric such that it conforms to a lumen in which the medical device is inserted to close any gaps between the device and the lumen in order to avoid stagnant pockets that could cause a thrombus to develop.

32. The coating of claim 1, further comprising a layer adjacent to the coating, wherein the layer comprises at least one anti-thrombogenic substance.

33. The coating of claim 1, further comprising an anti-thrombogenic substance.

34. The coating of claim 1, further comprising a layer adjacent to the coating, wherein the layer comprises at least one therapeutic agent for reducing clotting, selected from the group consisting of: heparin, ticlopidine, chlopidrel, enoxaparin, dalteparin, hirudin, dextran , bivalirudin, argatroban, danparoid, tissue factor pathway inhibitor (TFPI), a GPVI antagonist, an antagonist to a platelet adhesion receptor (GP1b-V-IX), and an antagonist to a platelet aggregation receptor (GPIIb-IIIa).

35. The coating of claim 1, further comprising at least one therapeutic agent for reducing clotting, selected from the group consisting of: heparin, ticlopidine, chlopidrel, enoxaparin, dalteparin, hirudin, dextran , bivalirudin, argatroban, danparoid, tissue factor pathway inhibitor (TFPI), a GPVI antagonist, an antagonist to a platelet adhesion receptor (GP1b-V-IX), and an antagonist to a platelet aggregation receptor (GPIIb-IIIa).

36. The coating of claim 1, further comprising a layer adjacent to the coating, wherein the layer comprises at least one endothelization promoting substance.

37. The coating of claim 36, wherein the endothelization promoting substance is selected from the group consisting of: vascular endothelial growth factor (VEGF), an antibody to CD34 receptors, angiopoietin-1, and phosphorylcholine.

38. The coating of claim 1, further comprising at least one endothelization promoting substance selected from the group consisting of: vascular endothelial growth factor (VEGF), an antibody to CD34 receptors, angiopoietin-1, and phosphorylcholine.

39. The coating of claim 1, further comprising a low density lipoprotein and/or a high density lipoprotein.

40. The coating of claim 1, wherein the medical device is a stent having struts, and the coating completely degrades in an amount of time it takes for the stent struts to be covered with intimal cells.

41. The coating of claim 2, wherein an amount of time for delaying onset of elution of the restenosis suppressing drug is from 5 days to 60 days.

42. The coating of claim 41, wherein the amount of time for delaying onset of elution of the restenosis suppressing drug is from 7 days to 45 days.

43. The coating of claim 42, wherein the amount of time for delaying onset of elution of the restenosis suppressing drug is from 15 to 30 days.

44. The coating of claim 41, wherein the amount of time for delaying onset of elution corresponds to:
an amount of time it takes for at least one drug-containing or drug-covering layer to degrade; and
an amount of time it takes for most of the medical device to become covered by a thin layer of cells produced by endothelization and/or restenosis.

45. The coating of claim 41, wherein the medical device is a stent having struts on its inside (or luminal surface) and further comprising more than one layer, wherein all layers collectively form the coating;
wherein the coating layers are arranged from the stent struts to an outermost surface of the stent in the following order:
(i) a primer layer;
(ii) a layer comprising at least one antiproliferative or immunosuppressant drug; and
(iii) a layer for delaying an onset of release of the antiproliferative or immunosuppressant drug.

46. A stent for implantation in a narrowed region of a blood vessel is coated with:
(i) a primer layer;
(ii) a layer comprising at least one antiproliferative or immunosuppressant drug; and
(iii) a layer comprising aligned elements for delaying an onset of release of the antiproliferative or immunosuppressant drug, wherein the aligned elements are selected from the group consisting of: nanofibers, microfibers, nanogrooves, microgrooves, and any combination of the aforementioned elements.

47. A method for delaying activation of an antiproliferative or an immunosuppressant drug around a medical device implanted within a body until after:
(i) 5-60 days have elapsed from medical device implantation; and
(ii) the medical device has been encapsulated by a thin layer of the body's own tissue.

48. The method of claim 47, wherein delaying activation is achieved at least in part by delaying elution of the drug from within the medical device, by providing a coating or a matrix, wherein the coating or the matrix degrades, erodes, and/or is absorbed by the body to expose the drug.

49. The method of claim 48, wherein the coating or the matrix comprises aligned fibers aligned grooves, or a combination of aligned fibers and aligned grooves.

50. The method of claim 49, wherein if there are fibers, at least some of the fibers are nanofibers, microfibers, or a combination of thereof or if there are grooves, at least some of the grooves are nanogrooves, microgrooves, or a combination thereof.

51. The method of claim 49, wherein the fibers or the grooves comprise a nitric oxide functional group and release nitric oxide as they degrade, erode, and/or are absorbed.

52. The method of claim 49, wherein the coating or the matrix further comprises at least one hydrophobic substance that breaks down quickly in a hydrophobic environment as provided by restenotic material.

53. The method of claim 52, further comprising a hydrophilic barrier positioned adjacent to the drug.

54. The method of claim 53, wherein the hydrophilic barrier comprises at least one element selected from the group consisting of: dextran, polyvinyl alcohol, polyethylene glycol (PEG, also known as poly(ethylene oxide) (PEO) or polyoxyethylene (POE)), gelatin, pullulan, heparin, chlopidogrel, a salt, and an anticoagulant.

55. The method of claim 53, wherein the hydrophobic substance comprises at least one element selected from the group consisting of: polylactide, polylactic acid, polyglycolide, polyglycolic acid, polylactide-polyglycolide, polycaprolactone, polyamino acid, and any copolymer of the aforementioned elements.

56. The method of claim 53, wherein the hydrophilic barrier is distributed in pockets within the hydrophobic coating or matrix.

57. The method of claim 56, wherein the hydrophobic coating or matrix has a higher viscosity than the hydrophilic barrier pockets.

58. The method of claim 56, wherein the hydrophilic barrier pockets repel the drug.

59. The method of claim 56, wherein the hydrophilic barrier pockets absorb water quickly upon degradation of the hydrophobic coating or matrix to form a low viscosity solution to facilitate drug elution.

60. The method of claim 59, wherein absorption of water by the hydrophilic barrier pockets upon initial degradation of the hydrophobic barrier coating or matrix causes water to flood the hydrophobic barrier, inducing its hydrolysis and accelerating further degradation of the hydrophobic barrier.

61. The method of claim 53, wherein the hydrophilic barrier consists of dextran and the hydrophobic coating or matrix consists of 75% polylactic acid and 25% polyglycolic acid.

62. The method of claim 47, wherein the drug is bound to a molecule that inactivates the drug until restenosis factors are present.

63. The method of claim 48, wherein the degradation, erosion, and/or absorption of the coating or the matrix is triggered by a restenosis factor selected from the group consisting of: a hormone, an enzyme, and a peptide.

64. The method of claim 48, wherein the degradation, erosion, and/or absorption of the coating or the matrix is triggered by a pH change accompanying restenosis.

65. The method of claim 48, wherein the degradation, erosion, and/or absorption of the coating or the matrix is triggered by a pressure change, beneath the coating or within the matrix, accompanying restenosis.

66. The method according to claim 48, wherein said coating or said matrix promotes endothelization by its geometry or by exposing blood to at least one endothelization promoting substance.

67. The method according to claim 48, wherein said coating or said matrix suppresses thrombus formation by its geometry or by exposing blood to one or more substance that suppresses thrombus formation.

68. A method of coating a drug eluting stent with tissue in vivo by intentionally allowing restenosis around the stent until a thin layer of tissue coats the stent.

69. The method according to claim 68, wherein the step of allowing restenosis is achieved by delaying release of one or more drug that would prevent restenosis from occurring over the drug eluting stent.

70. The coating of claim 2, wherein the restenosis suppressing drug is selected from the group consisting of: paclitaxel, rapamycin, sirolimus, everolimus, biolimus, zotarolimus, tacrolimus, fibroblast growth factor (bFGF), rapamycin analogs, antisense dexamethasone, angiopeptin, Batimistat™, Translast™, Halofuginon™, acetylsalicylic acid, Tranilast™, hirudin, steroids, ibuprofen, antimicrobials, antibiotics (including actinomycin D), tissue plasma activators, estradiol, and agents that affect VSMC (vascular smooth muscle cell) proliferation or migration (including transcription factor E2F1).
